(19) United States
(12) Patent Application Publication
(10) Pub. No.: US 2023/0201553 A1
(43) Pub. Date: Jun. 29, 2023

(54) Systems and Methods for Selective, Targeted Tissue Disruption

(71) Applicant: Insightec, Ltd., Tirat Carmel (IL)

(72) Inventors: Yoav Levy, Hinanit (IL); Eyal Zadicario, Tel-Aviv-Yafo (IL); Javier Grinfeld, Tel-Aviv-Yafo (IL); Rafi De Picciotto, Tirat Carmel (IL); Michael Plaksin, Tirat Carmel (IL); Avinoam Bar-Zion, Haifa (IL)

(21) Appl. No.: 18/176,978

(22) Filed: Mar. 1, 2023

Related U.S. Application Data
(63) Continuation-in-part of application No. 16/607,509, filed on Oct. 23, 2019.
(60) Provisional application No. 62/510,023, filed on May 23, 2017.

(51) Int. Cl. A61M 37/00; A61K 41/00

(52) U.S. Cl. CPC A61M 37/0092; A61K 41/0047; A61M 2210/0693; A61M 2205/3375

(57) ABSTRACT

Systems and methods for temporarily altering a tissue characteristic at a target region, such as the blood-brain barrier, include causing an ultrasound transducer to transmit acoustic energy to the target region at a transmission frequency; acquiring a cumulative harmonic response from at least the target region; and operating the transducer based at least in part on the acquired cumulative harmonic response.

### CROSS-REFERENCE TO RELATED APPLICATIONS

This is a continuation-in-part of U.S. Ser. No. 16/607,509, filed on Oct. 23, 2019, which claims priority to U.S. Provisional Patent Application No. 62/510,023, filed on May 23, 2017. The entire disclosures of the foregoing priority documents are hereby incorporated by reference.

### FIELD OF THE INVENTION

The field of the invention relates generally to ultrasound systems and, more particularly, to systems and methods for selective, targeted opening of the blood-brain barrier using an ultrasound procedure.

### BACKGROUND

The blood-brain barrier (BBB), formed by layers of cells in the central nervous system (CNS), excludes large molecules from entering the brain parenchyma, thereby protecting it from damage by toxic foreign substances. But the BBB also presents one of the largest obstacles to treating many brain diseases. Specifically, the BBB prevents many therapeutic agents, such as drugs and gene-therapy vectors, from reaching a patient's brain tissue. For example, treatments for CNS infections, neurodegenerative diseases, congenital enzyme defects and brain cancer are all hampered by the ability of the BBB to block passage of, inter alia, antibiotics, anti-retroviral drugs, enzyme replacement therapy, gene preparations and anti-neoplastic drugs. It is thus desirable to temporarily and locally “open” the BBB to permit therapeutic quantities of these agents to access the affected brain tissue.

Focused ultrasound (i.e., acoustic waves having a frequency greater than about 20 kilohertz) has been utilized to open the BBB in the treatment of neurological diseases. The mechanistic event underlying the BBB opening appears to involve the reaction of microbubbles to ultrasonic pulses, which can result in an array of behaviors known as acoustic cavitation. In stable cavitation, microbubbles expand and contract with the acoustic pressure rarefaction and compression over several cycles; such action can result in dilation and contraction of blood vessels in the vicinity. In inertial cavitation, the microbubbles can expand to several factors greater than their equilibrium radius and subsequently collapse due to the inertia of the surrounding tissue. In both cases, the consequent disruption of blood vessels induces “opening” of the BBB.

An uncontrolled microbubble cavitation, however, may result in undesired damage to and around the BBB. For example, ablating cells forming portions of the BBB may not only compromise BBB function but also cause unwanted cell death or necrosis in surrounding tissue. To minimize the undesired effects of microbubble cavitation during BBB disruption, one conventional approach utilizes a passive cavitation detector that measures the acoustic response of the microbubbles after each ultrasound sonication; if the acoustic response level is above a predefined threshold amplitude, the ultrasound procedure is suspended. The effects of microbubble cavitation, however, may be cumulative along a sonication and over a series of sonications, and may also depend on the properties of the tissue where cavitation occurs—that is, measuring the acoustic response from the microbubbles may not accurately reflect the effect of cavitation on the surrounding tissue.

Accordingly, there is a need to reliably detect microbubble cavitation resulting from ultrasound waves and monitor effects of the cavitation on the local tissue in real time so as to avoid permanent damage to the BBB or its surrounding tissue.

### SUMMARY

The present invention provides systems and methods for inducing microbubble cavitation with ultrasound in order to disrupt a target BBB region in a controlled and reversible manner. The formation and cumulative amount of microbubble cavitation may be reliably detected based on the measured acoustic response to delivered energy, i.e., the acoustic emissions produced by cavitation bubbles as measured by a detector (e.g., a passive cavitation detector, which may be an ultrasound transducer that can transmit or receive acoustic signals). Each ultrasound sonication pulse produces an acoustic response that may be measured, and the cumulative measurements over multiple sonications are herein referred to as the cumulative acoustic response. The physical work performed by the microbubbles in, for example, imparting energy to tissue so as to disrupt a barrier can only be measured indirectly, e.g., by observing the permeability of the BBB over time in the presence of cavitation. The relationship between acoustic energy delivered and work performed by the resulting cavitation may be established empirically by increasing the delivered acoustic energy and measuring the degree of tissue disruption as indicated, for example, by increased tissue permeability to an analyte of interest.

The cumulative acoustic response may be obtained as an integral of the time-varying acoustic response level over a predetermined time period. If the relationship between acoustic response and work done is known or can be estimated, progress in effecting tissue disruption as sonications are delivered can also be estimated. In addition, a safety limit can be defined to ensure that the magnitude and/or cumulative amount of microbubble cavitation that can be clinically tolerated—e.g., that does not permanently affect or damage the target BBB region or its surrounding tissue—is not reached; at that point, the ultrasound procedure may be suspended to avoid further inducing microbubble cavitation.

Assessing the effect of cavitation on tissue from the acoustic response can be challenging, since the correlation between work and the acoustic response signal is often complex. Microbubble oscillations manifest as emission signals with harmonic, sub-harmonic and ultraharmonic components due to the unique nonlinear, dynamic nature of the cavitation phenomenon. It is found, surprisingly, that the broadband response spectrum may reveal little about the amount of work exerted on tissue by cavitation. Rather, it is the harmonic and ultraharmonic responses, considered cumulatively, that correlate meaningfully with the tissue effect. Harmonic response analysis characterizes the response of a mechanical system at a specific frequency relative to the excitational acoustic energy delivered to the target—in particular, at a positive integer multiple of the applied acoustic frequency. The term “harmonic” means an integer multiple of the applied acoustic frequency and the term “ultraharmonic” means an off-integer multiple (e.g., 0.5, 1.5, 2.5, etc.) of the applied acoustic frequency; hence, for ease of presentation, “ultraharmonics” include sub-harmonics. The term “cumulative harmonic response” refers to the sum (for discrete measurements) or integral (for continuous “area under the curve” measurements) of signal amplitudes of the acoustic response at one or more harmonics and/or ultraharmonics of the applied acoustic signal.

In some embodiments, an imaging device (e.g., a magnetic resonance imaging (MRI) device) is employed to characterize tissue types and/or properties of the target BBB region and/or its surrounding tissue; each type and location of tissue, depending on its properties, may have corresponding tolerances for the acoustic response level and acoustic response. In addition, the imaging device may measure the cavitation effects (e.g., a temperature increase or an area that is disrupted) on the target BBB region and/or the surrounding tissue in real time. If an undesired effect on the target BBB region and/or its surrounding tissue is observed (e.g., the temperature increase exceeding a threshold and/or a disrupted area larger than a desired size), the ultrasound procedure may be halted. Accordingly, approaches described in the current invention may advantageously avoid permanent damage of the target BBB region and its surrounding tissue by reliably detecting microbubble cavitation events and monitoring effects of the cavitation on the target and/or surrounding tissue in real time.

Accordingly, in a first aspect, the invention pertains to a system for temporarily altering a tissue characteristic at a target region. In various embodiments, the system comprises an ultrasound transducer and a controller configured to cause the transducer to transmit acoustic energy to the target region at a transmission frequency, acquire a cumulative harmonic response from at least the target region, and operate the transducer based at least in part on the acquired cumulative harmonic response. For purposes hereof, operating based at least in part on the acquired cumulative harmonic response means based on the response from the target region, from around the target region, or from both target and non-target regions.

In some embodiments, the harmonic response is acquired at one or more positive integer multiples of the transmission frequency and/or at one or more positive off-integer multiples of the transmission frequency. The system may include a filter for filtering the measured acoustic signals from the target region and/or its surrounding regions to obtain the cumulative harmonic response. For example, the filter may be configured to select at least one of a harmonic, an ultraharmonic or a sub-harmonic response to the transmitted acoustic energy.

In some embodiments, the controller is further configured to compute the cumulative harmonic response by integrating a received acoustic signal from at least the target region over a predetermined time period. The controller may be configured to cause generation of microbubbles in the target region. In some embodiments, the system comprises an administration device for introducing microbubbles into at least one target region and/or one or more surrounding region.

In various embodiments, temporarily altering a tissue characteristic comprises or consists of disrupting the target tissue. For example, the target tissue may be BBB and the disruption may alter its permeability. The controller may be configured to control a parameter (such as power, frequency, pulse duration and/or pulse repetition frequency) of the transmitted acoustic energy based at least in part on spectral components of the cumulative harmonic response. The controller may be configured to control a parameter of the transmitted acoustic energy based at least in part on cumulative harmonic response data from within a defined interval. The interval may be within a current sonication. It may include data from at least one previous sonication.

In some embodiments, the controller is configured to control a parameter to select for a harmonic frequency band while maintaining cumulative broadband emission and/or cumulative ultra-harmonics below corresponding safety thresholds. Alternatively or in addition, the controller may be configured to control a parameter to increase the ratio between cumulative harmonics and cumulative ultra-harmonics, and/or between (i) cumulative harmonics and/or cumulative ultra-harmonics and (ii) cumulative broadband emission.

In another aspect, the invention relates to a method of applying ultrasound sonication from a transducer to temporarily alter a tissue characteristic at a target region. In various embodiments, the method comprises causing the transducer to transmit acoustic energy to the target region at a transmission frequency; acquiring a cumulative harmonic response from at least the target region; and operating the transducer based at least in part on the acquired cumulative harmonic response. In some embodiments, the cumulative harmonic response is acquired at one or more positive integer multiples of the transmission frequency and/or at one or more positive off-integer multiples of the transmission frequency. For example, the cumulative harmonic response may be acquired by integrating a received acoustic signal from at least the target region over a predetermined time period.

In some embodiments, the method further includes generating microbubbles in the target region. The method may include controlling a parameter of the transmitted acoustic energy based at least in part on spectral components of the cumulative harmonic response and/or based at least in part on cumulative harmonic response data from within a defined interval—e.g., within a current sonication or including data from at least one previous sonication. The method may include controlling a parameter to select for a harmonic frequency band while maintaining cumulative broadband emission and/or cumulative ultra-harmonics below corresponding safety thresholds. The method may include controlling a parameter to increase the ratio between cumulative harmonics and cumulative ultra-harmonics. In some embodiments, the method includes controlling a parameter to increase the ratio between (i) at least one of cumulative harmonics or cumulative ultra-harmonics and (ii) cumulative broadband emission. In all of these cases, the parameter may be power, frequency, pulse duration and/or pulse repetition frequency.

In still another aspect, the invention pertains to a method of applying a therapeutic agent to a brain tumor. In various embodiments, the method comprises causing the transducer to transmit acoustic energy to a target region including the BBB at a transmission frequency; acquiring a cumulative harmonic response from at least the target region; operating the transducer based at least in part on the acquired cumulative harmonic response to achieve a target level of BBB permeability; and administering the therapeutic agent to the target region at least when the target permeability level has been reached.

In various embodiments, the therapeutic agent comprises at least one of Busulfan, Thiotepa, CCNU (lomustine), BCNU (carmustine), ACNU (nimustine), Temozolomide, Methotrexate, Topotecan, Cisplatin, Etoposide, Irinotecan/SN-38, Carboplatin, Doxorubicin, Vinblastine, Vincristine, Procarbazine, Paclitaxel, Fotemustine, Ifosfamide/4-Hydroxyifosfamide/aldoifosfamide, Bevacizumab, 5-Fluorouracil, Bleomycin, Hydroxyurea, Docetaxel, or Cytarabine (cytosine arabinoside, ara-C)/ara-U, antibodies against tau or amyloid beta (e.g., Aducanamab), antibiotics and antivirals for combating CNS infections, an enzyme for enzyme replacement therapy or a gene preparation for gene therapy.

In yet another aspect, the invention relates to a method of treating a neurological disease or disorder in a subject in need thereof, wherein the neurological disease or disorder is characterized by having a locus of abnormal production, aggregation, and/or deposition of a protein or another biomolecule in the brain, the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached.

In various embodiments, the neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases. In some embodiments, the locus is selected from senile plaques, neurofibrillary tangles, neuronal inclusions, Lewy bodies, glial inclusions, cytoplasmic inclusions, and polyglutamine aggregates. In some embodiments, the protein showing abnormal production, aggregation, and/or deposition is selected from amyloid-β (AO), Tau protein, of TDP-43, α-Synuclein, FUS/TLS, SOD1, and Huntingtin.

In various embodiments, the therapeutic agent comprises a small molecule or a biologic drug. In some embodiments, the therapeutic agent is or comprises a biologic drug. In some embodiments, the therapeutic agent is selected from a gene therapy agent, a vaccine, an antisense oligonucleotide (ASO), a protein therapeutic, a modified mRNA agent, and a RNAi agent.

In some embodiments, the therapeutic agent is or comprises an antibody, antibody-like molecule or an antigen-binding fragment thereof. In some embodiments, the therapeutic agent specifically binds the protein or another biomolecule that that exhibits abnormal production, aggregation, and/or deposition. In some embodiments, the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), an anti-alpha-synuclein antibody (e.g., Cinpanemab, Prasinezumab, Lu AF82422, ABBV-0805, and MEDI1341), and or a combination thereof.

In some embodiments, the therapeutic agent is or comprises a small molecule drug. In some embodiments, the therapeutic agent provides one or more of synaptic plasticity, neuroprotection, reduction of inflammation, neurotransmitter receptor modulation, reduction of oxidative stress. In some embodiments, the therapeutic agent is selected from donepezil, galantamine, rivastigmine, memantine, suvorexant, carbidopa-levodopa, selegiline, rasagiline, safinamide, entacapone, benztropine, tolcapone, opicapone, nuplazid, istradefylline and amantadine, and a combination thereof.

In some embodiments, the therapeutic agent is formulated in a liposome. In some embodiments, the therapeutic agent is delivered via a viral vector.

As used herein, the term “substantially” means ±10 seconds, and in some embodiments, ±5 seconds. “Clinically tolerable” means having an undesired (and sometimes the lack of a desired) effect on tissue that is considered insignificant by clinicians, e.g., prior to triggering the onset of damage thereto. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

The foregoing will be more readily understood from the following detailed description of the invention in conjunction with the drawings, wherein:

FIG. 1 schematically depicts an exemplary ultrasound system in accordance with various embodiments of the current invention.

FIG. 2A depicts presence of microbubbles in a target tissue region in accordance with various embodiments.

FIGS. 2B-2D depict various configurations of the transducer elements performing a cavitation-detecting approach in accordance with various embodiments.

FIGS. 3A and 3B depict various relationships between an amplitude of a detected acoustic response level and a magnitude of the microbubble cavitation in accordance with various embodiments.

FIG. 3C depicts an exemplary relationship between a temporal acoustic response level and a cumulative acoustic response in accordance with various embodiments.

FIGS. 4A-4C illustrate amplitude variations of ultrasound pulses during an ultrasound procedure in accordance with various embodiments.

FIGS. 4D-4F illustrate various sonication patterns applied during an ultrasound procedure in accordance with various embodiments.

FIG. 5A depicts a relationship between a tissue response and a detected acoustic response level in accordance with various embodiments.

FIG. 5B depicts a relationship between a tissue response and a size of the target/non-target region disrupted by the microbubble cavitation in accordance with various embodiments.

FIG. 6 is a flow chart illustrating an approach of using ultrasound sonication and microbubbles to temporarily disrupt a patient's BBB in accordance with some embodiments of the present invention.

FIGS. 7A and 7B are spectra illustrating, respectively, the simulated and actual response of a passive cavitation detector to acoustic signals produced by oscillating microbubbles in cavitation.

FIG. 8 graphically illustrates the observed relationship between the work (e.g., tissue disruption) performed by microbubbles in cavitation and the resulting cumulative measured emissions.

FIG. 9 plots the observed increase in BBB permeability against the measured acoustic response at harmonic and ultraharmonic frequency bands in a pig model.

FIGS. 10A-10B show a series of plots, each corresponding to a different rat experiment, illustrating the observed increase in BBB permeability against the measured acoustic response at the second harmonic frequency band.

### DETAILED DESCRIPTION

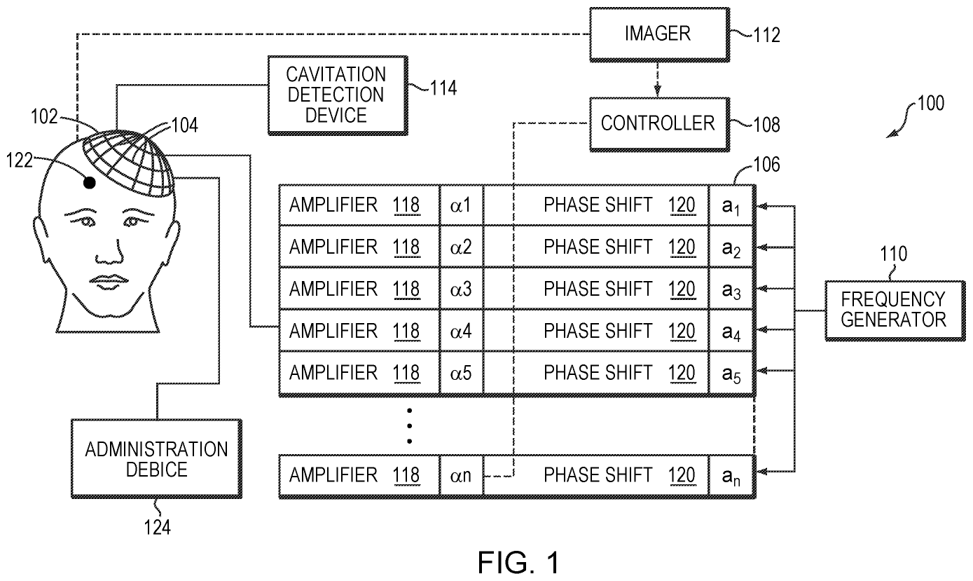

FIG. 1 illustrates an exemplary ultrasound system 100 for focusing ultrasound within a patient's brain (e.g., a target BBB region) through the skull. The applied ultrasound sonication may induce microbubble cavitation and disrupt the target BBB region in a controlled and reversible manner. In various embodiments, the system 100 includes a phased array 102 of transducer elements 104, a beamformer 106 driving the phased array 102, a controller 108 in communication with the beamformer 106, and a frequency generator 110 providing an input electronic signal to the beamformer 106. In various embodiments, the system further includes an imager 112, such as a magnetic resonance imaging (MRI) device, a computer tomography (CT) device, a positron emission tomography (PET) device, a single-photon emission computed tomography (SPECT) device, or an ultrasonography device, for determining anatomical characteristics of the skull, the target BBB region and the tissue surrounding the BBB region. The ultrasound system 100 and/or imager 112 may be utilized to detect the presence, type, and/or location associated with microbubble cavitation as further described below. Additionally or alternatively, in some embodiments, the system further includes a cavitation detection device (such as a hydrophone or suitable alternative) 114 to detect information associated with microbubble cavitation.

The array 102 may have a curved (e.g., spherical or parabolic) shape suitable for placing it on the surface of the skull, or may include one or more planar or otherwise shaped sections. Its dimensions may vary between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50Ω, matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each including or consisting of an amplifier 118 and a phase delay circuit 120; each drive circuit drives one of the transducer elements 104. The beamformer 106 receives a radio frequency (RF) input signal, typically in the range from 0.1 MHz to 1.0 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radio frequency generator 110 and the beamformer 106 are configured to drive the individual transducer elements 104 of the transducer array 102 at the same frequency, but at different phases and/or different amplitudes.

The amplification or attenuation factors α₁-αₙ and the phase shifts a₁-aₙ imposed by the beamformer 106 serve to transmit and focus ultrasonic energy through the patient's skull onto a selected region 122 of the patient's BBB, and account for wave distortions induced in the skull and soft brain tissue. The amplification factors and phase shifts are computed using the controller 108, which may provide the computational functions through software, hardware, firmware, hardwiring, or any combination thereof. For example, the controller 108 may utilize a general-purpose or special-purpose digital data processor programmed with software in a conventional manner, and without undue experimentation, in order to determine the phase shifts and amplification factors necessary to obtain a desired focus or any other desired spatial field patterns. In certain embodiments, the computation is based on detailed information about the characteristics (e.g., structure, thickness, density, etc.) of the intervening tissue (e.g., the skull and/or brain tissue) located between the transducer elements and the selected region 122 and their effects on propagation of acoustic energy. Such information may be obtained from the imager 112. Image acquisition may be three-dimensional or, alternatively, the imager 112 may provide a set of two-dimensional images suitable for reconstructing a three-dimensional image of the skull from which thicknesses and densities can be inferred. Image-manipulation functionality may be implemented in the imager 112, in the controller 108, or in a separate device.

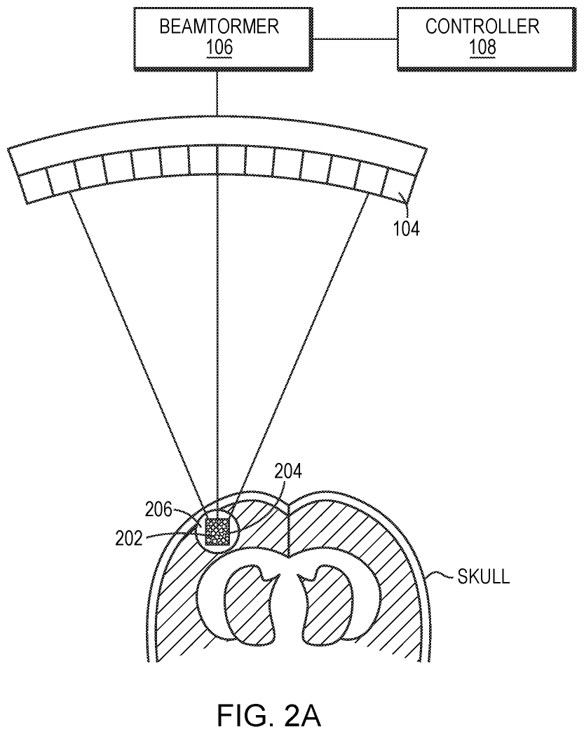

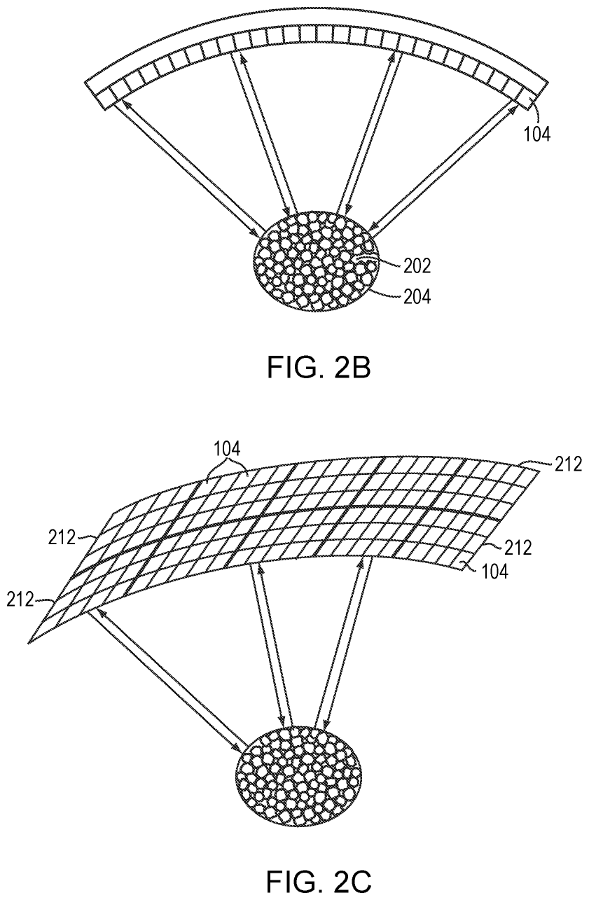

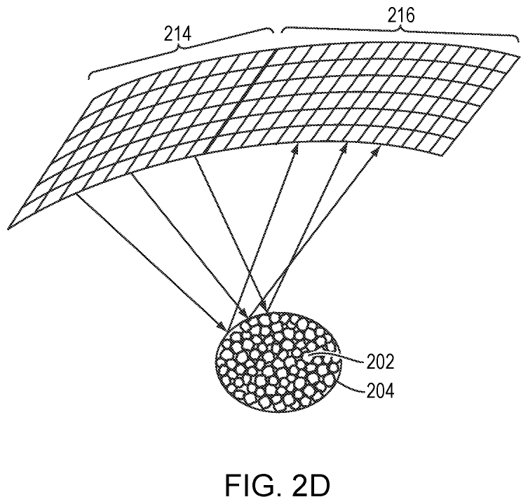

Referring to FIG. 2A, in various embodiments, the acoustic energy emitted by the transducer elements 104 may be above a threshold and thereby cause generation of a small cloud of gas bubbles (or “microbubbles”) 202 in the liquid contained in the target BBB region 204. The microbubbles 202 can be formed due to the negative pressure produced by the propagating ultrasonic waves or pulses, when the heated liquid ruptures and is filled with gas/vapor, or when a mild acoustic field is applied on tissue containing cavitation nuclei. Generally, at a relatively low acoustic power (e.g., 1-2 Watts above the microbubble-generation threshold), however, the generated microbubbles 202 undergo oscillation with compression and rarefaction that are equal in magnitude and thus the microbubbles generally remain unruptured. At a higher acoustic power (e.g., more than 10 Watts above the microbubble-generation threshold), the generated microbubbles 202 undergo rarefaction that is greater than compression, which may cause cavitation of the microbubbles. The microbubble cavitation may result in transient disruption (or “opening”) of the targeted BBB region 204, thereby allowing therapeutic or prophylactic agents present in the bloodstream to penetrate the “opened” BBB region 204 and effectively deliver therapy to the targeted brain cells.

Referring again to FIG. 1, in some embodiments, microbubbles are introduced into the patient's bloodstream, and may either be injected systemically into the patient's brain or locally into the target BBB region 204 using an administration system 124. For example, the microbubbles may be introduced into the patient's brain in the form of liquid droplets that subsequently vaporize, as gas-filled bubbles, or entrained with another suitable substance, such as a conventional ultrasound contrast agent. The injected microbubbles may themselves create or facilitate the creation of additional microbubbles. Therefore, the actual effect on the tissue may result from a combination of the injected microbubbles and microbubbles additionally created in the tissue. Approaches to generating the microbubbles and/or introducing the microbubbles to the target region are provided, for example, in U.S. Patent Application Nos. and 62/366,200, 62/597,071, Ser. Nos. 15/708,214, 15/837,392 and 62/597,073, the contents of which are incorporated herein by reference.

To avoid undesired damage of the target BBB region 204 and/or its surrounding tissue 206 resulting from the microbubble cavitation, in various embodiments, the formation and/or amount of induced microbubbles 202 in the target BBB region 204 is monitored by detecting acoustic signals emanating therefrom using the cavitation detection device 114, which then transmits the signals to the controller 108. Alternatively, the transducer elements 104 may possess both transmit and detect capabilities. Referring to FIG. 2B, in one embodiment, each individual transducer element 104 alternates between transmitting ultrasound signals to the microbubbles and receiving ultrasound signals therefrom. For example, all transducer elements 104 may substantially simultaneously transmit ultrasound to the microbubbles 202 and subsequently receive echo signals therefrom. Referring to FIG. 2C, in one implementation, the transducer array is divided into multiple sub-regions 212; each sub-region 212 comprises a one- or two-dimensional array (i.e., a row or a matrix) of transducer elements 104. The sub-regions 212 may be separately controllable, i.e., they are each capable of (i) emitting ultrasound waves/pulses at amplitudes, frequencies and/or phases that are independent of the amplitudes and/or phases of the other sub-regions 212, and (ii) measuring acoustic signals from the microbubbles 202. In one embodiment, the sub-regions 212 are assigned different amplitudes, frequencies and/or phases from one another, and activated, one at a time, to transmit ultrasound to and receive echo signals from the microbubbles 202. Referring to FIG. 2D, in another embodiment, the transducer array is divided into a transmit region 214 and a receive region 216; transducer elements in the transmit region 214 transmit the ultrasound waves/pulses while transducer elements in the receive region 216 receive the echo signals from the microbubbles 202. The received signals are then transmitted to the controller 108 for analysis. The transmit region 214 and receive region 216 of the transducer array may be configured in different patterns and shapes at various locations of the transducer array.

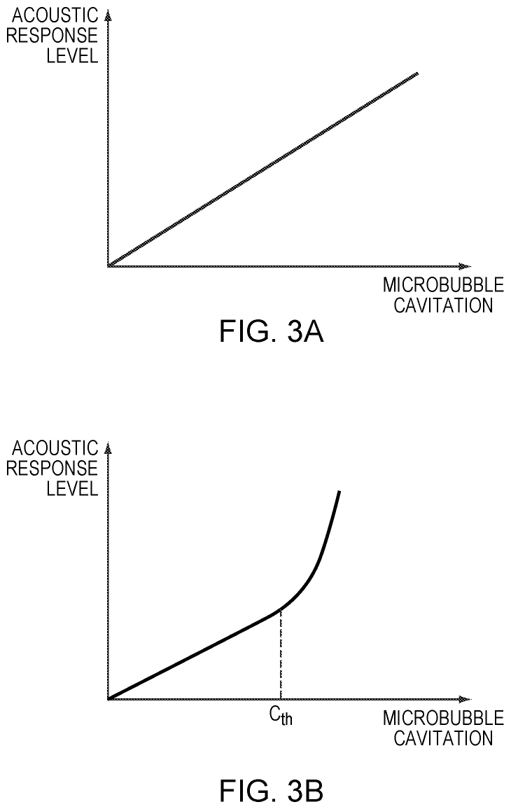

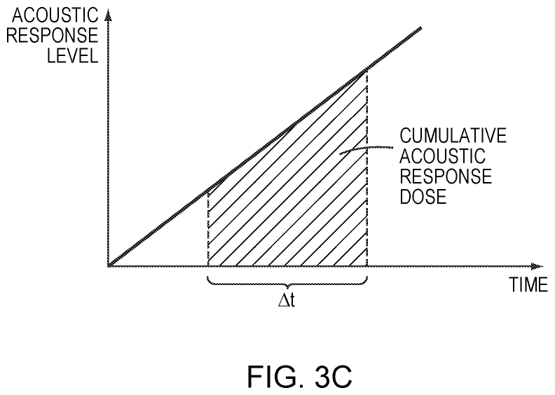

The microbubble acoustic signals may be emissions resulting from the shape change of the microbubbles 202 and/or reflections resulting from the microbubble encapsulating gas. The acoustic signals may include (i) an instantaneous acoustic response level and/or (ii) a spectral distribution of the acoustic response. The acoustic response level corresponds, either linearly or nonlinearly but in a known manner, to the magnitude of the acoustically driven cavitation. For example, referring to FIG. 3A, the amplitude of the detected acoustic response level may linearly correlate to the magnitude of the microbubble cavitation through the entire range thereof. Alternatively, referring to FIG. 3B, the linear correlation may occur only when the magnitude of the microbubble cavitation is below a threshold cavitation, Cₜₕ. When the magnitude of the cavitation exceeds the threshold, Cₜₕ, a small increase of the cavitation may result in a large increase of the response level of the acoustic signals.

The relationship between the amplitude of the acoustic response level and the magnitude of the microbubble cavitation may be empirically established from a pre-clinical study, a pre-treatment procedure, and/or from known literature. For example, in a pre-clinical study, the imager 112 may directly image the amount and/or area associated with the microbubble cavitation events, and based thereon, the magnitude of the microbubble cavitation may be quantified. Substantially simultaneously, the acoustic signals from the microbubble cavitation can be detected by the cavitation detection device 114 and/or transducer array 102 and subsequently analyzed by the controller 108 to acquire the amplitudes associated therewith. A relationship between the quantified magnitude of the microbubble cavitation and the amplitude of the acoustic response level can then be established.

In addition, the acoustic signals may include a spectral distribution of the acoustic response that indicates the type and/or location of the microbubble cavitation. This is because each type of the cavitation at each location may have its own spectral “signature” that represents the unique nonlinear response of the microbubbles. For example, the acoustic response of microbubbles may be linear at a relatively low acoustic power (e.g., 1-2 Watts above the microbubble-generation threshold); the spectral distribution of the detected signals may thus include a frequency that is the same as or a harmonic (or ultraharmonic or sub-harmonic) of that of the incident ultrasound waves. If stable cavitation is induced at an intermediate acoustic power (e.g., 5 Watts above the microbubble-generation threshold), the spectral distribution of the detected signals may include a strong sub-harmonic response (i.e., having more components at the sub-harmonic frequencies and/or having larger amplitudes of the sub-harmonic frequencies). Likewise, if inertial cavitation is induced at a high acoustic power (e.g., 10 Watts above the microbubble-generation threshold), the detected signals may include a broadband response. Thus, by detecting and analyzing the acoustic signals emitted from the microbubbles, the presence, type and/or of cavitation induced in tissue during an ultrasound procedure can be determined. Approaches to monitoring the cavitation events using signals from the microbubbles are provided, for example, in U.S. patent application Ser. No. 15/415,351, and the content of which is incorporated herein by reference.

In various embodiments, the detected spectral distribution of the acoustic response is filtered by one or more suitable filters implemented in hardware and/or software. For example, the filters may include multiple bandpass filters and/or window functions, each associated with a frequency component (e.g., the base frequency or one or more harmonics or ultraharmonics thereof) of the spectral signature. In one embodiment, the filters include a baseband filter that allows the baseband response of the signals to be processed. The filters may thus advantageously improve the resolution and/or signal-to-noise ratio of the detected signals, thereby allowing the presence, type and/or location of the microbubble cavitation to be reliably and accurately determined. Suitable filters are well-known in the art of signal processing (in particular, digital signal processing) and readily implemented without undue experimentation.

Alternatively or additionally, microbubble cavitation may be monitored using a cumulative acoustic response value that corresponds, either linearly or nonlinearly, to the cumulative cavitation-related acoustic energy imparted by the microbubbles over an entire sonication or over multiple successive sonication pulses. This is because the tissue tolerance may be a function both of the instantaneous response level and the cumulative cavitation-related response. For example, even if an instantaneous response level is below its corresponding predetermined threshold, the cumulative response may exceed its predetermined threshold; this may result in permanent effects or damage to the target BBB region or its surrounding tissue. Conversely, even if the cumulative response is below its predetermined threshold, a burst instantaneous response level above the threshold may be clinically intolerable. Accordingly, in a preferred embodiment, both the instantaneous response level and cumulative response are monitored during the ultrasound procedure.

Harmonic levels within the response signal are related to microbubble vibration—in particular, the type and magnitude of the vibration, which itself depends on the applied acoustic power. When vibrations are small, almost 100% of the response signal is in the transmission frequency band. Small vibrations stemming from low acoustic power levels and/or mismatch between the resonance frequency of the microbubbles (or bubble cloud) and the acoustic frequency, which results in low power transfer. As the vibration amplitude increases, other response modes appear in the measured signal. In experiments carried out in the human brain with a commercial contrast agent, the second harmonics typically appear first. At that stage, the vibrations usually start to affect the blood vessels and open the BBB but do not harm the vessels (i.e., microbleeding is generally not observed). At higher vibration amplitudes (where cavitation can occur), the emergence of ultraharmonics and sub-harmonics often coincides with damage to blood vessels and consequent microbleeds. Accordingly, controller 108 may consider the observed harmonic spectrum in effecting treatment and avoiding harm to the patient. For example, controller 108 may infer the onset of therapeutic tissue disruption upon the emergence of a harmonic regime (such as second harmonics) in the response signal, and may continue to increase applied acoustic power until harmonics indicative of possible tissue damage are detected in the spectral signature. In some embodiments, controller 108 is configured to control the applied power so as to increase ultra-harmonics while keeping the sub-harmonic band and the wideband below corresponding safety thresholds. Alternatively or in addition, controller 108 may be configured to control one or more parameters to select for the harmonic frequency band while maintaining cumulative broadband emission, cumulative ultra-harmonics or both below corresponding safety thresholds. In some embodiments, controller 108 controls one or more parameters to increase the ratio between cumulative harmonics and cumulative ultra-harmonics and/or the ratio between (i) at least one of cumulative harmonics or cumulative ultra-harmonics and (ii) cumulative broadband emission. The parameter(s) may be one or more of power, frequency, pulse duration or pulse repetition frequency.

In some embodiments, the cumulative acoustic response is defined utilizing the instantaneous acoustic response level. For example, referring to FIG. 3C, the cumulative acoustic response may be an integral of the acoustic response level over a predetermined time period, Δt; the predetermined time period may be the entire sonication procedure or over one or more successive sonication pulses. Accordingly, based on the received acoustic signals during the ultrasound procedure, the controller 108 may compute the acoustic response during any desired time period.

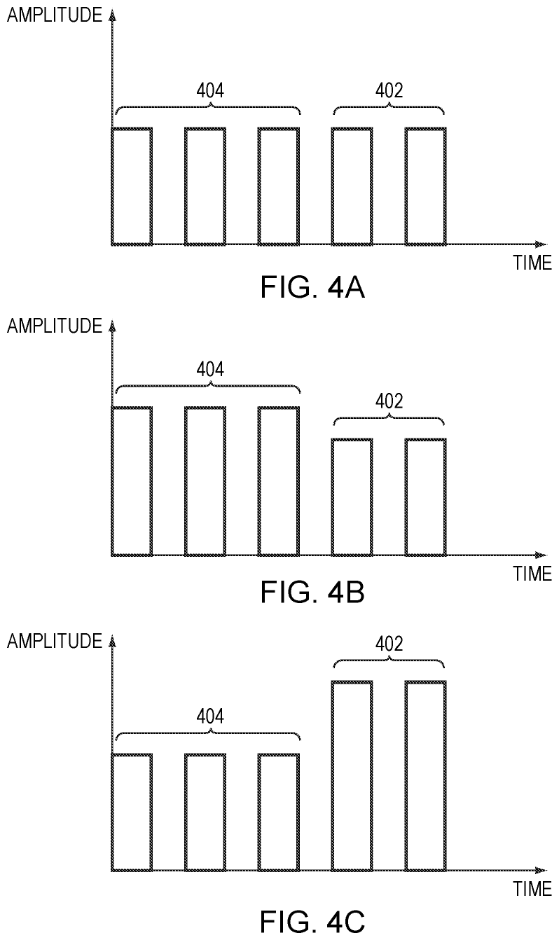

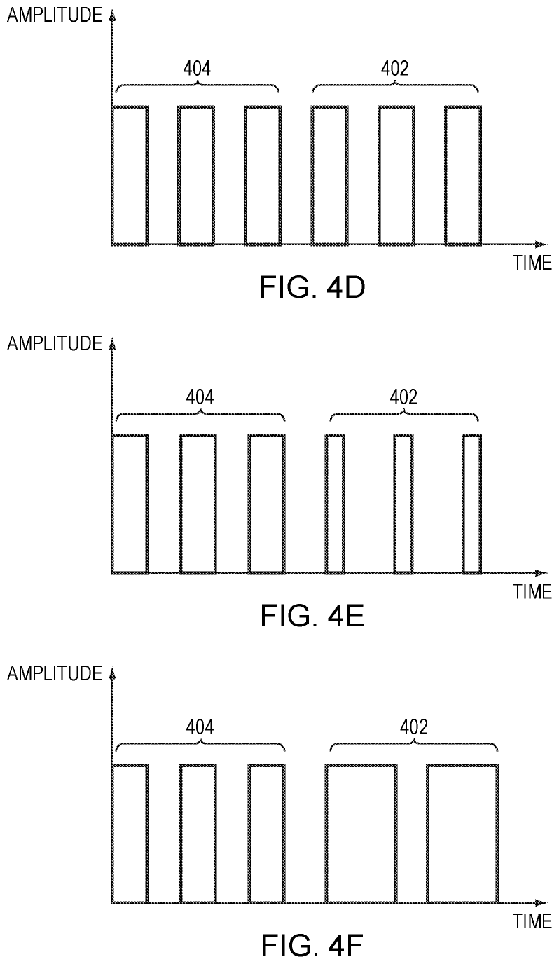

In addition, the detected acoustic response level and/or computed acoustic response may be compared with their associated predetermined threshold values stored in a database in memory; the threshold values represent an upper limit of the magnitude and/or amount of the microbubble cavitation that can be clinically tolerated. If the acoustic response level and/or acoustic response is at or above the predetermined threshold value, the ultrasound procedure may be suspended to avoid inducing more microbubble cavitation, thereby avoiding damage to the target and/or non-target tissue regions. If, however, the acoustic response level and/or acoustic response is below the corresponding predetermined threshold value, the ultrasound transducer elements 104 may deliver additional acoustic energy to the microbubbles so as to induce additional cavitation to disrupt the target BBB region. For example, referring to FIGS. 4A-4C, the transducer elements 104 may be activated to transmit one or more additional pulses 402 to the microbubbles; the amplitude of the additional pulse(s) 402 may be the same or different from that of the previous pulses 404. Alternatively or additionally, the sonication pattern (e.g., a frequency, a focusing shape, and/or a sonication profile varying with time) of the additional pulses 402 may be the same or different from that of the previous pulses 404. For example, a duty cycle of the elements' activation time in the additional pulses 402 may be the same, smaller than or larger than that in the previous pulses 404 as depicted in FIGS. 4D-4F. Generally, the duty cycles positively correlate to energy levels delivered to the microbubbles—a higher duty cycle corresponds to larger energy, because the power is on for most of the time.

It is possible to use the cumulative acoustic response for real-time control using frequent analysis of information extending back in time. For example, before each pulse within a sonication, the cumulative acoustic response over a previous defined time period (e.g., the previous five seconds) may be assessed to determine whether is within a range considered to be both safe and efficacious in terms of a treatment objective. If not, a parameter such as applied acoustic power may be adjusted, with the degree of adjustment evaluated and altered as necessary with each new assessment (based on the previous five-second window). This look-back period may be limited to the current sonication or may reach back into previous sonications (at least at the beginning of a sonication). If confined to the current sonication, the amount of temporal information available for control purposes increases through the sonication.

In some embodiments, when the acoustic response level and/or acoustic response is below the corresponding predetermined threshold value, additional microbubbles may be generated and/or introduced into the target BBB region in order to induce further microbubble cavitation. This can be achieved by activating the transducer array 102 to deliver more acoustic energy to the target BBB region and/or activating the administration system 124 to inject additional microbubbles into the target BBB region. In some embodiments, the administration device 124 first injects a seed microbubble into the target BBB region; the transducer array 102 then transmits an acoustic energy to the seed microbubble so as to generate more microbubbles.

The threshold values of the acoustic response level and cumulative acoustic response may be determined based on the tissue types, properties and/or other anatomical characteristics of the target BBB region and/or its surrounding region—the target BBB region and/or its surrounding region may include different types of tissue and/or have different tissue properties (e.g., densities, tolerance of thermal energy, thermal absorption coefficients, etc.) and thereby respond differently to the ultrasound pulses and/or microbubble cavitation; consequently, thresholds of the acoustic response level and acoustic response may differ for different types of tissue at different locations. In addition, the threshold values of the acoustic response level and cumulative acoustic response may depend on other parameters associated with an ultrasound treatment protocol, sonication pattern (e.g., the frequency, duty cycle, focusing shape, and/or sonication profile varying with time) and/or history of the acoustic and/or tissue response during the current or previous treatments. For example, lower threshold values may be used for ultrasound pulses that have a higher duty cycle; this is because the target/non-target tissue may have less time to relax between consecutive pulses. In some embodiments, larger threshold values are tolerable at the beginning of the treatment but smaller thresholds are preferred after, for example, the occurrence of a major therapeutic event during the treatment.

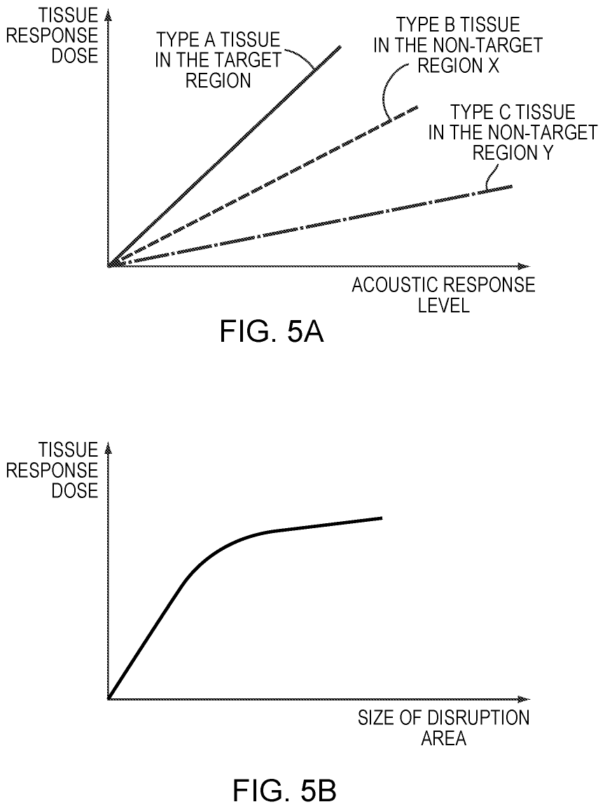

In various embodiments, operation of the transducer elements 104 (such as activation, deactivation or adjustment of the sonication pattern) and/or the administration system 124 is determined based on a tissue response. The tissue response may be based on the maximum clinically tolerable temperature for each affected target/non-target region based on the types, properties and/or other anatomical characteristics of the tissue in each region. Thus, various type of tissue having different properties at different locations may have different tissue responses. The tissue response may be obtained using any suitable approach prior to and/or during treatment. For example, referring to FIG. 5A, the tissue response may be empirically correlated to the acoustic response level and/or cumulative acoustic response; accordingly, the tissue response may be acquired based on the acoustic response level and/or cumulative acoustic response detected/computed as described above.

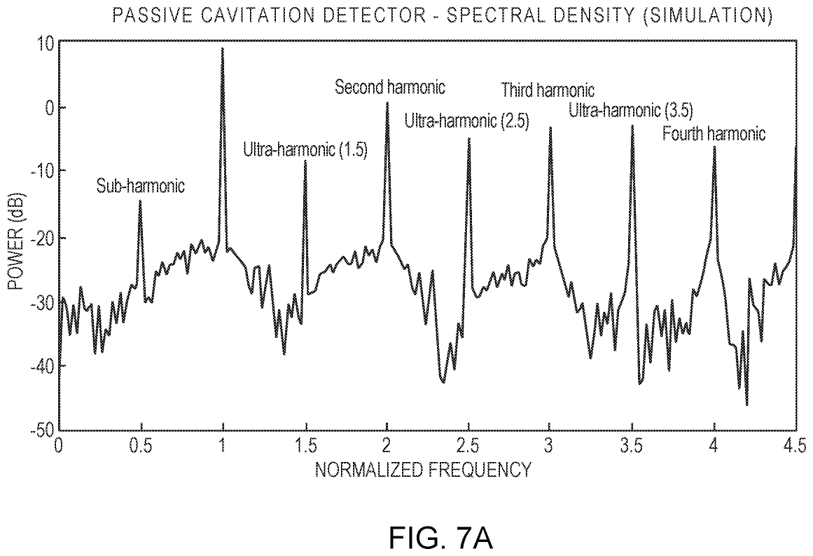

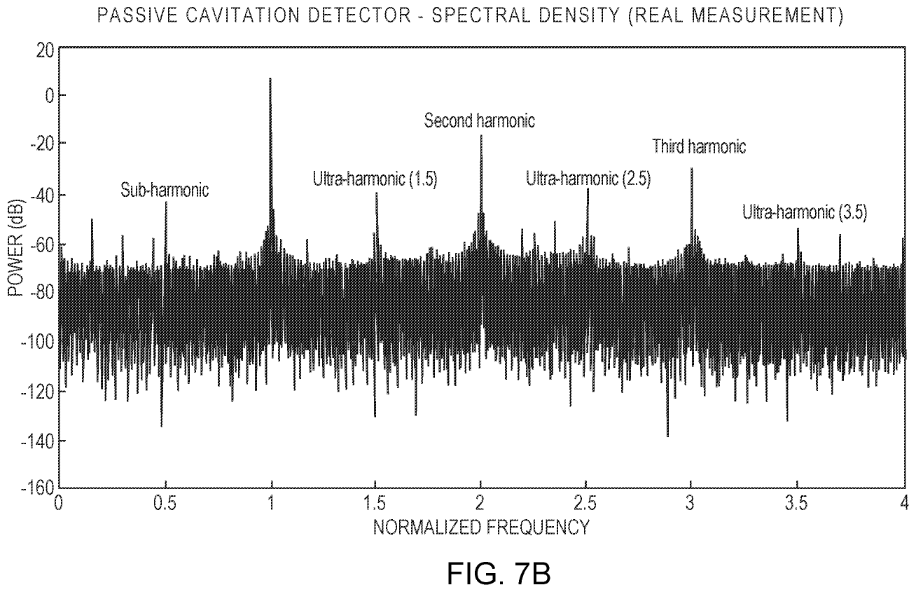

It is found that harmonics and ultraharmonics in the detected cavitation-related signal are well-correlated to, and therefore indicative of, the “work” performed by microbubbles in cavitation. The dynamics of the behavior of microbubbles in cavitation are well represented by the Rayleigh-Plesset equation, which may be used to simulate the emission spectrum as shown in FIG. 7A. The simulation is based on a bubble radius of 3 mm and a sonication frequency of 230 kHz. Amplitude peaks are predicted to occur at harmonics and ultraharmonics of the applied sonication frequency, and although the response spectrum of a real system as shown in FIG. 7B is noisier than the simulation, the peaks are clearly seen.

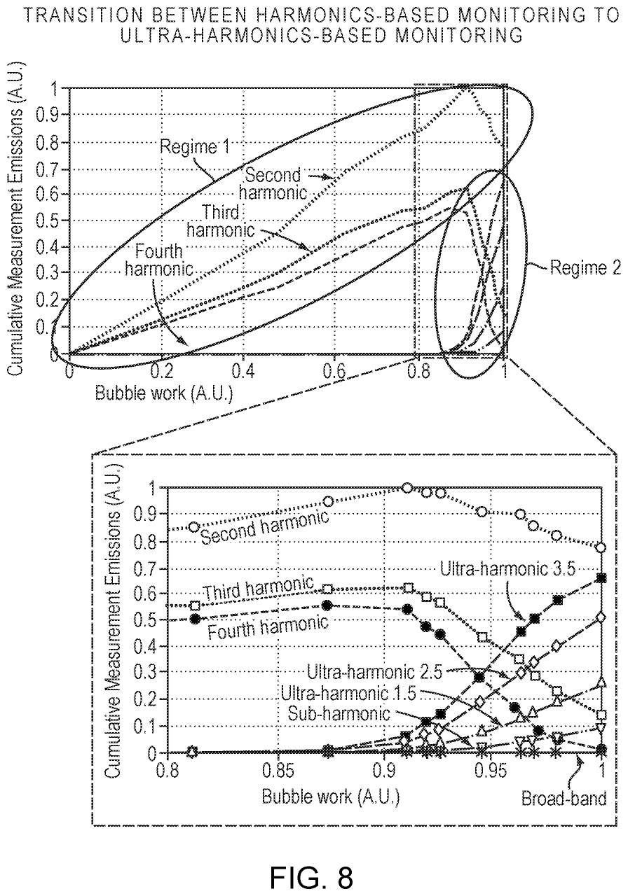

FIG. 8 illustrates the simulated relationship between the cumulative harmonic response (in arbitrary units, A.U.) in various harmonic and ultraharmonic spectral bands and the work performed by cavitation effects, i.e., energy transfer from cavitation bubbles to the surrounding medium. This corresponds, for example, to disrupting tissue such as the BBB in order to, e.g., increase the permeability thereof. The plots are based on a Rayleigh-Plesset simulation of the behavior of microbubbles having a 3 mm radius under exposure to a 230 kHz ultrasound frequency field. Regime 1 shows a rising monotonic correlation between the cumulative harmonic response and work. In this regime, no correlation between work and broadband and ultraharmonic (including subharmonic) bands are seen. That is, below 0.8 A.U., only received signals in the harmonic signal bands are predictive of cavitation work performed. Regime 2 shows a rising monotonic correlation between the cumulative ultraharmonic (including subharmonic) response and work performed; thus, above 0.8 A.U., only signals in the ultraharmonic bands are predictive of cavitation work performed. Notably, the broadband signal is never meaningfully predictive.

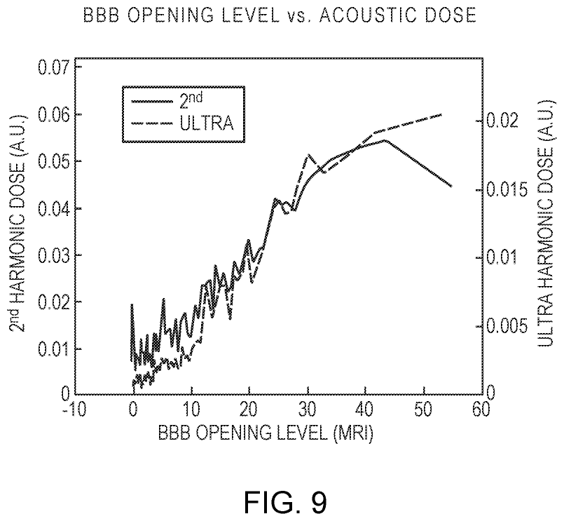

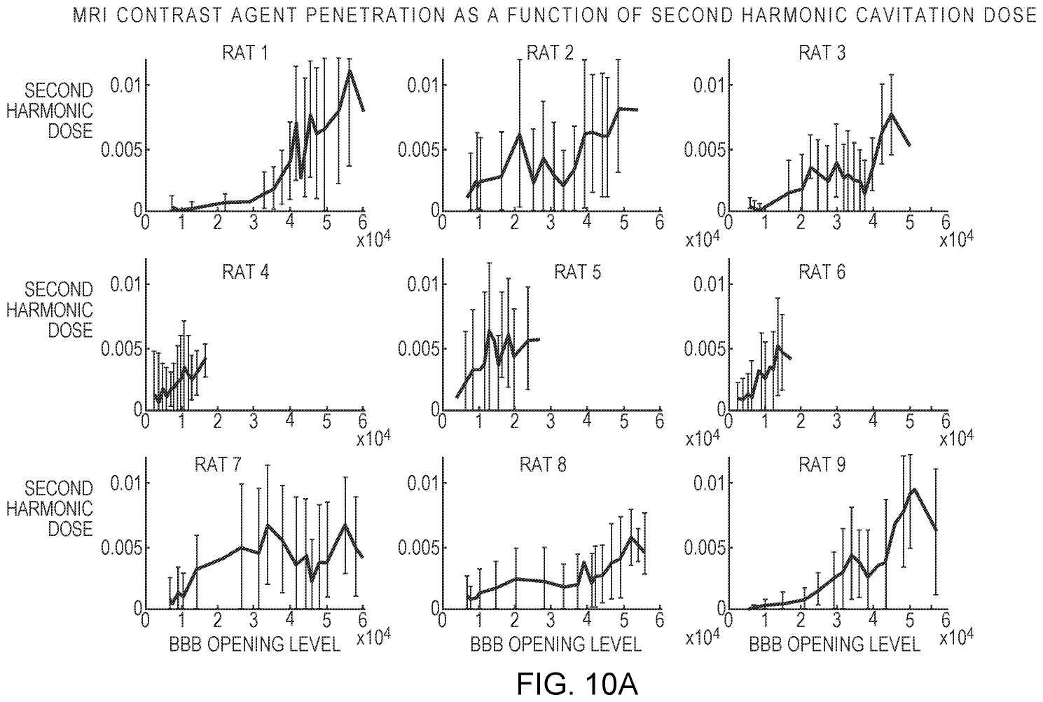

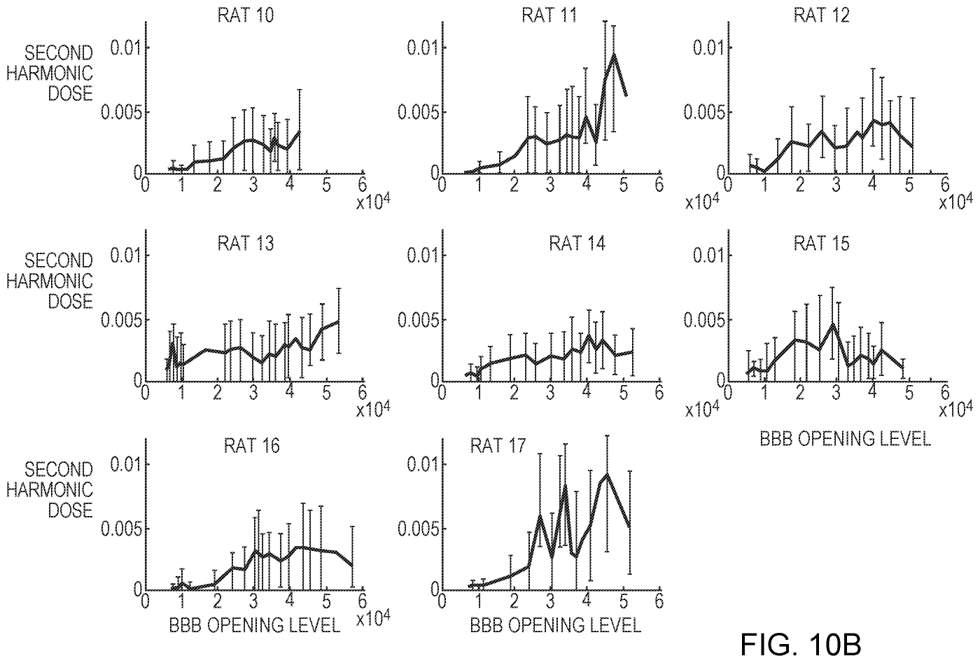

FIG. 9 explicitly illustrates the relationship between BBB permeabilization (due to work performed thereon) and the cumulative second harmonic and ultraharmonic (in particular, 2.5 times the sonication frequency) response. Both responses correlate well with the observed permeability. Similarly, as shown in FIGS. 10A-10B, a predictive relationship between the cumulative second harmonic response and BBB permeability is observed across numerous experiments conducted on rats. BBB permeability was assessed by measuring the penetration of an MRI contrast agent.

It should be noted that the acoustic beam usually causes more than one microbubble to undergo cavitation. Since microbubbles are not identical to each other, measurements in the second regime reflect the fact that some microbubbles likely have previously passed into this regime and exploded. As a result, the effect on tissue in Regime 2 is relatively more aggressive. In addition, all the work done by the cavitating bubbles on surrounding tissue depends on the type of tissue, which may dictate the preferred regime. For example, white matter, which has a relatively low vascular density (and seems to be more sensitive in BBB-related treatments), may be good candidate for Regime 1. In general, sensitive regions (such as subregions of the gray matter) can be good candidates for this regime as well.

Changes in the concentration of microbubbles during treatment can also affect the decision to work in one regime or the other. For example, when the bubble concentration is high and more bubbles perform work, the harmonic regime may be preferred, whereas when the concentration is low, the ultraharmonic regime may be preferred since fewer bubbles perform work and larger bubble oscillations are needed to produce a desired tissue effect. Finally, although the previous discussion focused on microbubbles, similar results can be expected with bubbles in different size ranges (e.g., nanobubbles) and phase droplets.

Additionally or alternatively, the tissue response may be determined using the imager (e.g., an MRI device) 112. For example, the MRI device 112 may measure the disrupted area of the target BBB region 204 and/or its surrounding region 206 resulting from the microbubble cavitation in real time. The size of the disrupted area may correlate to the tissue response as shown in FIG. 5B. In various embodiments, the temperature at the target BBB region and/or it surrounding region represents the tissue response thereof. The temperature dependence of MRI T₂ relaxation times in the target BBB region 204 and its surrounding region 206 may be determined prior to the ultrasound procedure. During the ultrasound procedure, the MRI T₂ relaxation time can be measured quickly (within 1 ms to 1 sec) in order to estimate the temperature of the target BBB region 204 and/or it surrounding region 206 in real time. This approach advantageously provides real-time temperature feedback to the ultrasound controller 108 which may adjust the ultrasound transmission accordingly. For example, if the measured temperature is below the predetermined maximum temperature, the ultrasound procedure may continue to induce more microbubble cavitation to disrupt the target BBB region 204 by, for example, transmitting additional pulses, increasing the amplitudes and/or duty cycles of the pulses. If, however, the measured temperature is above the maximum temperature, the ultrasound procedure may be halted to avoid permanently damaging the tissue. It should be noted that other MRI signals may be additionally or alternatively used to estimate the tissue response of the target BBB region 204 and/or it surrounding region 206. For example, MRI T₂* weighted imaging may advantageously detect an extravasation (level) of the blood in the brain. In addition, temperature-sensitive MR parameters, such as the proton resonance frequency (PRF), the diffusion coefficient (D), T₁ and T₂ relaxation times, magnetization transfer, and/or the proton density, as well as temperature-sensitive contrast agents, may be utilized alone or in combination to estimate the tissue response.

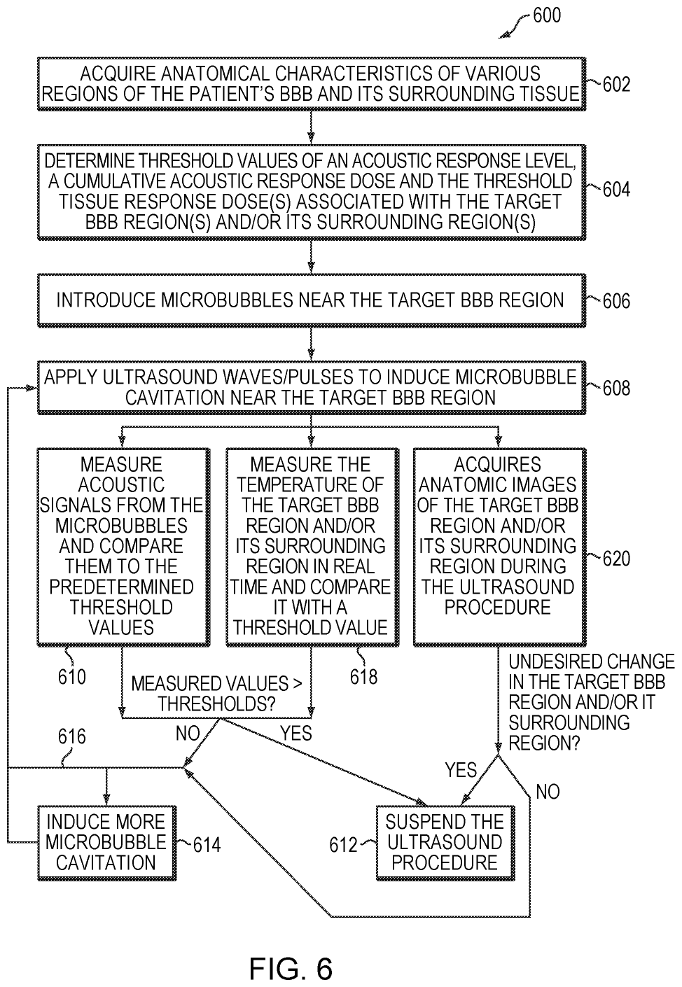

FIG. 6 illustrates a representative approach 600 to using ultrasound sonication to induce microbubble cavitation for temporarily disrupting a patient's BBB in a controlled and reversible manner. In a first step 602, an imager (e.g., an MRI device) is utilized to acquire anatomical characteristics of various regions of the patient's BBB and its surrounding tissue prior to applying the ultrasound sonication. In a second step 604, threshold values of an acoustic response level and a cumulative acoustic response associated with the microbubbles at each one of the target BBB regions and/or its surrounding region(s), and threshold tissue response(s) associated with the target BBB region(s) and/or its surrounding region(s) may be determined based on the anatomical characteristics of the tissue extracted from the MRI data as described above. Different types of the target/non-target tissue at different locations may have different threshold values. The threshold values of the acoustic response level and cumulative acoustic response and the threshold tissue response(s) may then be stored in a database in memory that can be accessed by the controller 108. In a third step 606, microbubbles are generated by application of the ultrasound pulses and/or introduced by an administration system near the target BBB region. In a fourth step 608, the ultrasound transducer array may be activated to apply waves or pulses so as to induce microbubble cavitation near the target BBB region. In a fifth step 610, acoustic signals from the microbubbles may be continuously measured (e.g., after delivery of each sonication) and compared to the predetermined threshold values of the acoustic response level and/or cumulative acoustic response. If the amplitudes of the detected acoustic signals are above the threshold values, the magnitude and/or amount of microbubble cavitation may exceed a safe (i.e., clinically tolerable) level; therefore, the ultrasound procedure may be suspended until the detected acoustic signal amplitudes fall below the threshold values or after a recovery interval following application of the maximum tolerable dose (in a sixth step 612).

If the acoustic response level and/or cumulative acoustic response are below the respective threshold values, more microbubbles may be generated and/or introduced to increase cavitation events to continue disruption of the target BBB region (in a seventh step 614). Additionally or alternatively, the ultrasound transducer may be activated to deliver the next wave/pulse with the same or different amplitude and sonication pattern from the previous applied waves/pulses (in an eighth step 616). In addition, during the ultrasound procedure, the imager (e.g., MRI device) may measure the temperature of the target BBB region and/or its surrounding region in real time (in a ninth step 618). For example, the real-time temperature may be acquired by measuring the MRI T₂ relaxation time. Again, if the measured temperature is below the predetermined threshold of the tissue response, additional microbubbles may be generated and/or introduced (step 614) and/or the ultrasound procedure may continue (step 616). If, however, the measured temperature is above the threshold, the ultrasound procedure is halted to avoid overheating which may result in permanent damage to the target BBB region and/or its surrounding region (step 612). In one embodiment, the database may alternatively or additionally store threshold values associated with other temperature sensitive MR parameters, such as the PRF, diffusion coefficient (D), T₁ relaxation time, magnetization transfer, proton density, as well as parameters associated with the temperature sensitive contrast agents. The imager may then measure these parameters during the ultrasound procedure; the measured values may then be compared against the stored threshold values and, based thereon, the controller 108 may operate the transducer array 102 and/or administrative system as described above.

In some embodiments, the MRI device also acquires anatomic images of the target BBB region and/or its surrounding region during the ultrasound procedure (in a tenth step 620). If an undesired change in the target BBB region and/or its surrounding region is observed, the ultrasound procedure may be stopped immediately. The undesired change may include, for example, the size of the disrupted BBB area being larger than the desired area and/or a portion of the non-target surrounding region being disrupted. Embodiments of the present invention thus employ a cavitation detection device (or an ultrasound transducer array) and an imaging device to monitor formation/generation of the microbubbles, the cavitation events, and tissue response in real-time during an ultrasound procedure; based on the monitored response, disruption of the target BBB region may then be facilitated in a controlled manner without permanently damaging the target BBB region and/or its surrounding region.

Thereafter, a therapeutic agent may penetrate from the bloodstream to the targeted brain cells via the opened BBB region. The therapeutic agent may include any drug that is suitable for treating a brain tumor. For example, for treating glioblastoma (GBM), the drug may include or consist of, e.g., one or more of Busulfan, Thiotepa, CCNU (lomustine), BCNU (carmustine), ACNU (nimustine), Temozolomide, Methotrexate, Topotecan, Cisplatin, Etoposide, Irinotecan/SN-38, Carboplatin, Doxorubicin, Vinblastine, Vincristine, Procarbazine, Paclitaxel, Fotemustine, Ifosfamide/4-Hydroxyifosfamide/aldoifosfamide, Bevacizumab, 5-Fluorouracil, Bleomycin, Hydroxyurea, Docetaxel, Cytarabine (cytosine arabinoside, ara-C)/ara-U, etc.

Those skilled in the art can select a drug and a BBB opening regime optimized to enhance drug absorption across the BBB within patient safety constraints. In this regard, it is known that the BBB is actually already disrupted in the core of many tumors, allowing partial penetration of antitumor drugs; but the BBB is widely intact around the “brain adjacent to tumor” (BAT) region where invasive/escaping GBM cells can be found, and which cause tumor recurrence. Overcoming the BBB for better drug delivery within the tumor core and the BAT can be accomplished using ultrasound as described herein. The drugs employed have various degrees of toxicity and various penetration percentages through the BBB. An ideal drug has high cytotoxicity to the tumor and no BBB penetration (so that its absorption and cytotoxic effects can be confined to regions where the BBB is disrupted), low neurotoxicity (to avoid damage to the nervous system), and tolerable systemic toxicity (e.g., below a threshold) at the prescribed doses. The drug may be administered intravenously or, in some cases, by injection proximate to the tumor region.

Functionality for performing disruption of a target BBB region in a controlled and reversible manner as described above, whether integrated within the controller 108 of the ultrasound system 100, the imager 122 and/or the administration system 124 or provided by a separate external controller, may be structured in one or more modules implemented in hardware, software, or a combination of both. In addition, the imager 122 and/or the administration system 124 may be controlled by the controller 108 or other separate processor(s). For embodiments in which the functions are provided as one or more software programs, the programs may be written in any of a number of high level languages such as PYTHON, FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80x86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

The following discussion describes neurological disease or disorders that may be treated using the methods and systems disclosed herein.

In some aspects, the present disclosure provides a method of treating a neurological disease or disorder in a subject in need thereof, wherein the neurological disease or disorder is characterized by abnormal production, aggregation, and/or deposition of a protein or another biomolecule in the brain. In some embodiments, neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases.

Alzheimer's Disease patients exhibit senile plaques that are mainly composed of amyloid-β (Aβ), neurofibrillary tangles, which include Tau protein, neuronal inclusions of TDP-43 as well as Lewy bodies, which include α-Synuclein. Parkinson's Disease patients exhibit Lewy bodies, which include α-Synuclein. Patients suffering from amyotrophic lateral sclerosis have neuronal inclusions that include TAR DNA-binding protein 43 (TDP-43), fused in sarcoma/translocated in liposarcoma (FUS/TLS), and superoxide dismutase-1 (SOD1). Huntington's Disease is a progressive brain disorder caused by a mutation in the gene coding for the huntingtin protein, resulting in an abnormal mutant protein that gradually damages brain cells. Dementia with Lewy bodies features Lewy bodies, which include α-Synuclein, senile plaques that are mainly composed of amyloid-β (Aβ) and neurofibrillary tangles of Tau protein. The patients having frontotemporal diseases show neuronal and glial inclusions composed of Tau, TDP-43, and FUS/TLS. Multiple system atrophy features glial cytoplasmic inclusions of α-synuclein. Thus, there appears to be an overlap between the proteins that exhibits abnormal production, aggregation, and/or deposition associated with these diseases. A single neurodegenerative disease can be associated with multiple proteins (or another biomolecules) that exhibits abnormal production, aggregation, and/or deposition. On the other hand, a single the protein that exhibits abnormal production, aggregation, and/or deposition can also be associated with multiple diseases. For example, although Aβ plaques and tau tangles are paradigmatic of Alzheimer's Disease, Lewy bodies typical of Parkinson's Disease are found in more than 50 percent of Alzheimer's cases, and neuronal inclusions consisting of the protein TDP-43 are found in more than 40 percent. Similarly in dementia with Lewy bodies, a dementing disorder closely allied to Parkinson's disease having some features of Alzheimer's, the paradigmatic α-Synuclein-rich Lewy bodies are accompanied by Aβ plaques in 60 percent of cases and tau tangles in 50 percent. Likewise, four-repeat tauopathies, a group of neurodegenerative diseases defined by cytoplasmic inclusions predominantly composed of tau protein isoforms with four microtubule-binding domains, is associated with at least three clinical presentations: (1) progressive supranuclear palsy presents with an axial rigidity and eye movement problems, in addition to atypical Parkinsonism; (2) corticobasal degeneration presents like a frontal lobe dementia, with focal cortical syndromes, including progressive apraxia or progressive aphasia; and (3) argyrophilic grain disease is an increasingly recognized disorder of the elderly that affects the medial temporal lobe and is associated with an amnesic cognitive impairment.

Accordingly, in some aspects, the present disclosure provides a method of treating a neurological disease or disorder in a subject in need thereof, wherein the neurological disease or disorder is characterized by abnormal production, aggregation, and/or deposition of a protein or another biomolecule in the brain, the method comprising: (a) selecting the subject having a locus of abnormal production, aggregation, and/or deposition of the protein in the brain; (b) identifying a region of blood-brain barrier (BBB) for an ultrasound treatment, wherein the region is adjacent to or fully encompassing the locus; (c) administering a therapeutic agent to the subject; and (d) applying an ultrasound beam across the cranium of the subject to the region of BBB to facilitate transit of the therapeutic agent across BBB to the locus. In some embodiments, the neurological disease or disorder is Alzheimer's Disease and the locus is selected from a senile plaque comprising amyloid-β (Aβ), neurofibrillary tangles comprising Tau protein, neuronal inclusions comprising TDP-43, and Lewy bodies comprising α-Synuclein. In some embodiments, the neurological disease or disorder is Parkinson's Disease and the locus is Lewy bodies comprising α-Synuclein. In some embodiments, the neurological disease or disorder is amyotrophic lateral sclerosis and the locus is a neuronal inclusion comprising TAR DNA-binding protein 43 (TDP-43), fused in sarcoma/translocated in liposarcoma (FUS/TLS), and superoxide dismutase-1 (SOD1). In some embodiments, the neurological disease or disorder is Huntington's Disease and the locus is neuronal intranuclear inclusions of Huntingtin. In some embodiments, the neurological disease or disorder is dementia with Lewy bodies and the locus is Lewy bodies comprising α-Synuclein, senile plaques comprising amyloid-β (Aβ), and neurofibrillary tangles comprising Tau protein. In some embodiments, the neurological disease or disorder is frontotemporal diseases and the locus is neuronal and glial inclusions composed of Tau, TDP-43, and FUS/TLS. In some embodiments, the neurological disease or disorder is multiple system atrophy, and the locus is glial cytoplasmic inclusions of α-synuclein. In some embodiments, the neurological disease or disorder is four-repeat tauopathy, and the locus is cytoplasmic inclusions predominantly composed of tau protein isoforms with four microtubule-binding domains.

The diseases that are associated with aggregation and/or accumulation of the protein (or another biomolecule) that exhibits abnormal production, aggregation, and/or deposition also include prion diseases, i.e., the transmissible spongiform encephalopathies such as bovine spongiform encephalopathy (BSE or mad cow disease) and Creutzfeldt-Jakob disease. These diseases feature senile plaques made of PrP protein. Accordingly, in some aspects, the present disclosure provides a method of treating a prion disease, the method comprising: (a) selecting the subject having a locus of deposition of PrP protein in the brain; (b) identifying a region of blood-brain barrier (BBB) for an ultrasound treatment, wherein the region is adjacent to or fully encompassing the locus; (c) administering a therapeutic agent to the subject; and (d) applying an ultrasound beam across the cranium of the subject to the region of BBB to facilitate transit of the therapeutic agent across BBB to the locus.

Alzheimer's Disease

Alzheimer's disease (AD) is a complex, progressively debilitating, and fatal neurodegenerative disease. AD is rapidly increasing in frequency as the world's population ages. There are currently an estimated 6.5 million individuals with AD in the US, and this number is expected to increase to more than 13 million by 2050. Approximately 15% of the US population over age 60 has prodromal AD and approximately 40% has preclinical AD. Similar trends are seen globally with an anticipated worldwide population of AD dementia patients exceeding 100 million by 2050 unless means of delaying, preventing, or treating AD are found. There is a significant need for therapeutics that halt or reverse the underlying pathology of AD.

Cellular and molecular mechanisms of AD are not well understood yet. Researchers have been reported that AD is associated with genetic and environmental factors and life-style. AD patients are heterogeneous in that they could be in preclinical AD continuum spanning up to two decades or more without exhibiting any clinical symptoms, i.e., mild cognitive impairment (MCI), AD dementia, or functional decline. Furthermore, misdiagnosis of AD patients is common in that 10-30% of individuals clinically diagnosed as AD dementia do not display AD neurodegeneration at autopsy.

Across all types of AD therapies, the failure rate is more than 99%, and for disease-modifying therapies (DMTs), the failure rate is 100%. Therefore, in addition to new approaches for developing therapeutic agents, approaches, such as those disclosed herein, for targeted delivery of the therapeutic agents is required.

Alzheimer's Disease is associated senile plaques composed of amyloid-β (Aβ), neurofibrillary tangles, which include Tau protein, neuronal inclusions of TDP-43 as well as Lewy bodies, which include α-Synuclein. Aβ is a relatively small peptide of 4 to 4.4 kDa that is the major component of amyloid deposits. Intracellular Aβ protein is widely found in neurons and it is associated with inflammatory and antioxidant activity, regulation of cholesterol transport, and activation of kinase enzyme. However, Aβ is one of the best known components in formation of neurodegenerative diseases including AD. Aβ is approximately composed of 36-43 amino acids and it originates from amyloid precursor protein (APP), which is a glycoprotein of 695-770 amino acids. APP can be cleaved into fragments by α, β, and γ secretases and Aβ protein is formed by the action of the β and γ secretases. Aβ protein contains two important regions which play a major role in the formation insoluble amyloid fibrils.

The microtubule associated Tau protein, the name of which is derived from “tubulin associated unit,” is highly expressed in brain. Microtubules are major proteins of the cytoskeleton. The main function of the Tau protein is to stabilize microtubules with binding to microtubules and to other proteins. To perform these functions, Tau protein is phosphorylated at normal level. Hyperphosphorylation of Tau protein is believed to cause conformational changes and aggregation of tau proteins. Other post-translational modifications such as glycosylation, glycation, polyamination, and nitration may play roles in aggregation. Accordingly, in some aspects, the present disclosure provides a method of treating Alzheimer's Disease (AD), the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is selected from amyloid-β peptide (Aβ), neurofibrillary tangles and tau protein. In some embodiments, the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), donepezil, rivastigmine, memantine and galantamine and a combination thereof. In some embodiments, the therapeutic agent is aducanumab. Some therapeutic agents are disclosed in WO 2014/089500 and WO 2021/108861, and the content of which is incorporated herein by reference.

Parkinson's Disease

Parkinson's disease (PD) is a long-term degenerative disorder of the central nervous system that causes unintended or uncontrollable movements, such as shaking, stiffness, and difficulty with balance and coordination. Symptoms usually begin gradually and worsen over time. As the disease progresses, people may have difficulty walking and talking. They may also have mental and behavioral changes, sleep problems, depression, memory difficulties, and fatigue. The occurrence of the illness is characterized by accumulation of misfolded α-synuclein protein in brain. Generally; anxiety, tremor, rigidity, depression, bradykinesia, and postural abnormalities are the most common symptoms in Parkinson's disease.

Lewy bodies (LBs), which mainly consist of α-syn, are neuropathological hallmarks of patients with Parkinson's disease (PD). It has been increasingly recognized, however, that PD is frequently associated with cognitive deficits, and that dementia eventually develops in a substantial number of patients.

α-synuclein is associated with a number of neurodegenerative diseases that are known as “Synucleinopathies.” Natively unfolded α-synuclein (α-Syn) is a 14 kDa and highly conserved protein that localize different regions of the brain. The name of protein was preferred as “α-synuclein” because of it shows synaptic and nuclear localization. α-Syn regulates dopamine neurotransmission by modulation of vesicular dopamine storage. It interacts with tubulin and can function like tau protein. Also, α-Syn shows a molecular chaperon activity in folding of SNARE (soluble N-ethylmaleimide-sensitive-factor attachment protein receptor) proteins. α-Syn plays crucial role in PD because α-Syn is a major fibrillary component for Lewy bodies. Two mutations, A53T and A30P, in the α-Syn gene and overexpression of wild type α-Syn are increases misfolding processes and aggregation. Also, accumulation of abnormal form of α-Syn can inhibit proteasomal functions. In PD brains, α-Syn is found to be phosphorylated at Ser87 and Ser129 in aggregates. These serine residues are phosphorylated with casein kinase 1 (CK1) and casein kinase 2 (CK2). It is believed that this post translational modification has a pathological role in fibrillation of α-Syn.

Accordingly, in some aspects, the present disclosure provides a method of treating Parkinson's Disease (PD), the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is alpha-synuclein. In some embodiments, the therapeutic agent is selected from an anti-alpha-synuclein antibody (e.g., Cinpanemab, Prasinezumab, Lu AF82422, ABBV-0805, and MEDI1341), carbidopa-levodopa, selegiline, rasagiline, safinamide, entacapone, benztropine, tolcapone, opicapone, nuplazid, istradefylline and amantadine, and a combination thereof.

Multiple system atrophy features glial cytoplasmic inclusions of α-synuclein. Accordingly, in some aspects, the present disclosure provides a method of treating multiple system atrophy, the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is alpha-synuclein. In some embodiments, the therapeutic agent is selected from an anti-alpha-synuclein antibody (e.g., Cinpanemab, Prasinezumab, Lu AF82422, ABBV-0805, and MEDI1341), carbidopa-levodopa, selegiline, rasagiline, safinamide, entacapone, benztropine, tolcapone, opicapone, nuplazid, istradefylline and amantadine, and a combination thereof

Dementia with Lewy Bodies

Dementia with Lewy bodies features Lewy bodies, which include α-Synuclein, senile plaques that are mainly composed of amyloid-β (Aβ) and neurofibrillary tangles of Tau protein. Dementia with Lewy bodies (DLB) is a type of progressive dementia that leads to a decline in thinking, reasoning and independent function. Its features may include spontaneous changes in attention and alertness, recurrent visual hallucinations, REM sleep behavior disorder, and slow movement, tremors or rigidity. Mutations in genes known as SNCA and SNCB can cause dementia with Lewy bodies. Mutations in another gene called GBA or a certain version of a gene called APOE increase the risk of developing the condition, but are not a direct cause. Accordingly, in some aspects, the present disclosure provides a method of treating dementia with Lewy bodies, the method comprising (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is alpha-synuclein. In some embodiments, the therapeutic agent is selected from an anti-alpha-synuclein antibody (e.g., Cinpanemab, Prasinezumab, Lu AF82422, ABBV-0805, and MEDI1341), rivastigmine, donepezil, galantamine, memantine, carbidopa-levodopa, and a combination thereof. In some embodiments, the methods disclosed herein further comprises detecting a brain-derived biomarker in a plasma sample of the subject. In some embodiments, the method detects a mutation in a gene selected from SNCA, SNCB, and APOE.

Huntington's Disease

Huntington's disease (HD) is a genetic neurodegenerative disorder and the disease is caused by autosomal dominant inheritance. HD patients show involuntary muscle contractions, movement, and mental disorders. The disease is inherited as an autosomal dominant and effects brain and nervous systems. Huntington protein undergoes conformational changes with mutation and it shows aggregation tendency.

In HD, the neuropathology is characterized with accumulation of Htt protein aggregates. HD is caused by a number of CAG repeats in the gene. It is believed that the CAG repeats (polyQ) are the most important promoter for toxicity of Htt protein aggregates. The polyQ region starts at residue 18 and the number of glutamine residues are the most important marker in HD. Surprisingly, 40 or more CAG repeats are always generated neuropathy, while 35 or fewer CAG repeats are never generated neuropathy. However, in childhood, CAG repeats from 27 to 35 can develop neuropathy. Accordingly, in some aspects, the present disclosure provides a method of treating Huntington's Disease (HD), the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is huntingtin. In some embodiments, the therapeutic agent is selected from anti-huntingtin antibody and an anti-SEMA4D antibody (e.g., Pepinemab).

Amyotrophic Lateral Sclerosis (ALS)

Amyotrophic lateral sclerosis (ALS) is a fatal neurodegenerative disorder characterized by degeneration of both upper and lower motor neurons, leading to progressive paralysis in muscles of the limbs, speech, swallowing and respiration. Patients suffering from amyotrophic lateral sclerosis have neuronal inclusions that include TAR DNA-binding protein 43 (TDP-43), fused in sarcoma/translocated in liposarcoma (FUS/TLS), and superoxide dismutase-1 (SOD1). ALS pathology is believed to begin at a single focal or multifocal sites and spread through the neuroaxis in a spatiotemporal manner. Insoluble TDP-43 from diseased brains has been reported to induce TDP-43 pathology in neuroblastoma cells that overexpress wtTDP-43 as detected by TDP-43 hyperphosphorylation, ubiquitination and aggregation. Accordingly, in some aspects, the present disclosure provides a method of treating amyotrophic lateral sclerosis (ALS), the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is TAR DNA-binding protein 43 (TDP-43), fused in sarcoma/translocated in liposarcoma (FUS/TLS), and superoxide dismutase-1 (SOD1). In some embodiments, the therapeutic agent is selected from an anti-TDP-43 antibody, an anti-SOD1 antibody, riluzole, edaravone, and sodium phenylbutyrate and taurursodiol, or a combination thereof

Spinocerebellar Ataxia

Spinocerebellar ataxia (SCAs) is a complex group of neurodegenerative disorders characterized by progressive cerebellar ataxia of gait and limbs variably associated with ophthalmoplegia, pyramidal and extrapyramidal signs, dementia, pigmentary retinopathy and peripheral neuropathy. Disease onset is usually between 30 and 50 years of age, although early onset in childhood and onset in later decades after 60 years have been reported. The prognosis is variable depending on the underlying cause of the spinocerebellar ataxia subtype. Mutations in ATXN1, ATXN2, ATXN3, SCA4, SPTBN2, CACNAIA, ATXN7, KLHL1AS, ATXN10, SCA11, PPP2R2B, KCNC3, PRKCG, etc. are found in SCAs. In addition, seven spinocerebellar ataxia subtypes including SCAs 1, 2, 3/Machado-Joseph disease, 6, 7, 17 and dentatorubral pallidoluysian atrophy (DRPLA) are caused by the expansion of a CAG-repeat sequence in specific genes, leading to abnormally long polyQ tracts in the encoded proteins. Proteins with expanded stretches of polyglutamine appear to take on an abnormal configuration resulting in the formation and deposition of polyglutamine aggregates in disease neurons forming characteristic nuclear or cytoplasmic inclusions, which are neuropathological hallmarks in these diseases. Accordingly, in some aspects, the present disclosure provides a method of treating spinocerebellar ataxia, the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is a mutant protein having expanded polyglutamine tracts. In some embodiments, the therapeutic agent is selected from an anti-polyglutamine antibody

Frontotemporal Diseases

The clinical syndromes of frontotemporal dementia are clinically and neuropathologically heterogeneous, but processes such as neuroinflammation may be common across the disease spectrum. In recent years, attention has focused on understanding the pathogenic role of protein misfolding and aggregation, which is a cardinal feature of the post-mortem diagnostic criteria for frontotemporal lobar degeneration (FTLD). These diseases are associated neuronal and glial inclusions composed of Tau, TDP-43, and FUS/TLS. Frontotemporal dementia with parkinsonism-17 (FTDP-17) is a progressive neurodegenerative disease which is caused by mutations in the tau gene. The tau gene is mutated in familial FTDP-17 and this mutation accelerates formation of neurofibrillary tangles (NFTs) in the brain. Furthermore, hyperphosphorylation is promoted by this mutation. In some aspects, the present disclosure provides a method of treating frontotemporal dementia, the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the locus is neuronal and glial inclusions composed of Tau, TDP-43, and FUS/TLS. In some embodiments, the therapeutic agent is an anti-tau antibody.

Four-Repeat Tauopathy

Four-repeat (4R-) tauopathies are a group of neurodegenerative diseases defined by cytoplasmic inclusions of tau protein isoforms. Progressive supranuclear palsy, corticobasal degeneration, argyrophilic grain disease or glial globular tauopathy belong to the group of 4R-tauopathies. Tau is a microtubule-associated protein with versatile functions in the dynamic assembly of the neuronal cytoskeleton, and in these diseases, cytoplasmic inclusions predominantly composed of tau protein isoforms with four microtubule-binding domains are found. Moreover, Tau protein is generally located in axons, but in tauopathy, it is located in dendrites. Thus, neuron's transport system may be disintegrated and microtubule cannot function correctly. Accordingly, in some aspects, the present disclosure provides a method of treating a four-repeat (4R-) tauopathy, the method comprising: (a) causing an ultrasound transducer to transmit acoustic energy to a target region at a transmission frequency, wherein the target region encompasses the locus and adjacent blood-brain barrier (BBB); (b) acquiring a cumulative harmonic response from at least the target region; (c) operating the transducer based at least in part on the acquired cumulative harmonic response to achieve a target level of BBB permeability; and (d) administering a therapeutic agent to the target region at least when the target permeability level has been reached. In some embodiments, the protein that exhibits abnormal production, aggregation, and/or deposition is Tau protein. In some embodiments, the therapeutic agent is an anti-tau antibody.

1. A system for temporarily altering a tissue characteristic at a target region, the system comprising:
an ultrasound transducer; and
a controller configured to:
(a) cause the transducer to transmit acoustic energy to the target region at a transmission frequency;
(b) acquire a cumulative harmonic response from at least the target region; and
(c) operate the transducer based at least in part on the acquired cumulative harmonic response.

2. The system of claim 1, wherein the harmonic response is acquired at one or more positive integer multiples of the transmission frequency.

3. The system of claim 1, wherein the harmonic response is acquired at one or more positive off-integer multiples of the transmission frequency.

4. The system of claim 1, further comprising a filter for filtering the measured acoustic signals from the target region and/or its surrounding regions to obtain the cumulative harmonic response.

5. The system of claim 4, wherein the filter is configured to select at least one of a harmonic, an ultraharmonic or a sub-harmonic response to the transmitted acoustic energy.

6. The system of claim 1, wherein the controller is further configured to compute the cumulative harmonic response by integrating a received acoustic signal from at least the target region over a predetermined time period.

7. The system of claim 1, wherein the controller is further configured to cause generation of microbubbles in the target region.

8. The system of claim 1, further comprising an administration device for introducing microbubbles into at least one of the target region or its surrounding regions.

9. The system of claim 1, wherein temporarily altering a tissue characteristic comprises disrupting the target tissue.

10. The system of claim 9, wherein the target tissue is the blood-brain barrier (BBB) and the disruption alters a permeability of the BBB.

11. The system of claim 1, wherein the controller is configured to control a parameter of the transmitted acoustic energy based at least in part on spectral components of the cumulative harmonic response.

12. The system of claim 11, wherein the parameter is at least one of power, frequency, pulse duration or pulse repetition frequency.

13. The system of claim 11, wherein the controller is configured to control a parameter of the transmitted acoustic energy based at least in part on cumulative harmonic response data from within a defined interval.

14. The system of claim 13, wherein the interval is within a current sonication.

15. The system of claim 13, wherein the interval includes data from at least one previous sonication.

16. The system of claim 11, wherein the controller is configured to control a parameter to select for a harmonic frequency band while maintaining at least one of cumulative broadband emission or cumulative ultra-harmonics below corresponding safety thresholds.

17. The system of claim 11, wherein the controller is configured to control a parameter to increase a ratio between cumulative harmonics and cumulative ultra-harmonics.

18. The system of claim 11, wherein the controller in configured to control a parameter to increase a ratio between (i) at least one of cumulative harmonics or cumulative ultra-harmonics and (ii) cumulative broadband emission.

19. A method of applying ultrasound sonication from a transducer to temporarily alter a tissue characteristic at a target region, the method comprising:
(a) causing an ultrasound transducer to transmit acoustic energy to the target region at a transmission frequency;
(b) acquiring a cumulative harmonic response from at least the target region; and
(c) operating the transducer based at least in part on the acquired cumulative harmonic response.

20. The method of claim 19, wherein the cumulative harmonic response is acquired at one or more positive integer multiples of the transmission frequency.

21. The method of claim 19, wherein the cumulative harmonic response is acquired at one or more positive off-integer multiples of the transmission frequency.

22. The method of claim 19, wherein the cumulative harmonic response is acquired by integrating a received acoustic signal from at least the target region over a predetermined time period.

23. The method of claim 19, further comprising generating microbubbles in the target region.

24. The method of claim 19, further comprising the step of controlling a parameter of the transmitted acoustic energy based at least in part on spectral components of the cumulative harmonic response.

25. The method of claim 24, wherein the parameter is at least one of power, frequency, pulse duration or pulse repetition frequency.

26. The method of claim 24, further comprising the step of controlling a parameter of the transmitted acoustic energy based at least in part on cumulative harmonic response data from within a defined interval.

27. The method of claim 26, wherein the interval is within a current sonication.

28. The method of claim 27, wherein the interval includes data from at least one previous sonication.

29. The method of claim 24, further comprising the step of controlling a parameter to select for a harmonic frequency band while maintaining at least one of cumulative broadband emission or cumulative ultra-harmonics below corresponding safety thresholds.

30. The method of claim 24, further comprising the step of controlling a parameter to increase a ratio between cumulative harmonics and cumulative ultra-harmonics.

31. The method of claim 24, further comprising the step of controlling a parameter to increase a ratio between (i) at least one of cumulative harmonics or cumulative ultra-harmonics and (ii) cumulative broadband emission.

32. The method of claim 19, further comprising introducing microbubbles into at least one of the target region or its surrounding regions.

33. A method of treating brain cancer in a subject in need thereof, the method comprising:
(a) causing an ultrasound transducer to transmit acoustic energy to a target region including the blood-brain barrier (BBB) at a transmission frequency;
(b) acquiring a cumulative harmonic response from at least the target region;
(c) operating the transducer based at least in part on the acquired cumulative harmonic response to achieve a target level of BBB permeability; and
(d) administering a therapeutic agent to the target region at least when the target permeability level has been reached.

34. The method of claim 33, wherein brain cancer is selected from glioblastoma (GBM), Diffuse Intrinsic Pontine Glioma (DIPG), brain metastases of lung cancer, breast cancer, colon cancer, kidney cancer, lung cancer, melanoma, glioma, astrocytoma, sububependymoma, ependymoma, myxopapillary ependymoma, glioblastoma, oligoastrocytoma and oligodendroglioma, meningioma, acoustic neuroma (schwannoma), pituitary adenoma, medulloblastoma, hemangiopericytoma, pineal tumor, chordoma, chondrosarcoma, olfactory neuroblastoma (esthesioneuroblastoma), gliosarcoma, lymphoma, rhabdomyosarcoma, paranasal sinus cancer, atypical teratoid/rhabdoid tumor, or craniopharyngioma.

35. The method of claim 33, wherein the therapeutic agent comprises at least one of Busulfan, Thiotepa, CCNU (lomustine), BCNU (carmustine), ACNU (nimustine), Temozolomide, Methotrexate, Topotecan, Cisplatin, Etoposide, Irinotecan/SN-38, Carboplatin, Doxorubicin, Vinblastine, Vincristine, Procarbazine, Paclitaxel, Fotemustine, Ifosfamide/4-Hydroxyifosfamide/aldoifosfamide, Bevacizumab, 5-Fluorouracil, Bleomycin, Hydroxyurea, Docetaxel, or Cytarabine (cytosine arabinoside, ara-C)/ara-U.

36. The method of claim 33, further comprising introducing microbubbles into at least one of the target region or its surrounding regions.

37. A method of treating a neurological disease or disorder in a subject in need thereof, wherein the neurological disease or disorder is characterized by having a locus of abnormal production, aggregation, and/or deposition of a protein or another biomolecule in the brain, the method comprising:
(a) causing an ultrasound transducer to transmit acoustic energy to a target region at a transmission frequency, wherein the target region encompasses the locus and adjacent blood-brain barrier (BBB);
(b) acquiring a cumulative harmonic response from at least the target region;
(c) operating the transducer based at least in part on the acquired cumulative harmonic response to achieve a target level of BBB permeability; and
(d) administering a therapeutic agent to the target region at least when the target permeability level has been reached.

38. The method of claim 37, wherein the neurological disease or disorder is selected from the Alzheimer's Disease (AD), Parkinson's Disease (PD), Huntington's Disease (HD), amyotrophic lateral sclerosis (ALS), dementia with Lewy bodies, spinocerebellar ataxia, and amyotrophic lateral sclerosis, frontotemporal diseases, multiple system atrophy, four-repeat tauopathy and prion diseases.

39. The method of claim 37, wherein the locus is selected from senile plaques, neurofibrillary tangles, neuronal inclusions, Lewy bodies, glial inclusions, cytoplasmic inclusions, and polyglutamine aggregates.

40. The method of claim 37, wherein the protein showing abnormal production, aggregation, and/or deposition is selected from amyloid-β (Aβ), Tau protein, of TDP-43, α-Synuclein, FUS/TLS, SOD1, and Huntingtin.

41. The method of claim 37, wherein the therapeutic agent comprises a small molecule or a biologic drug.

42. The method of claim 41, wherein the therapeutic agent is or comprises a biologic drug.

43. The method of claim 42, wherein the therapeutic agent is selected from a gene therapy agent, a vaccine, an antisense oligonucleotide (ASO), a protein therapeutic, a modified mRNA agent, and a RNAi agent.

44. The method of claim 42, wherein the therapeutic agent is or comprises an antibody, antibody-like molecule or an antigen-binding fragment thereof.

45. The method of claim 44, wherein the therapeutic agent specifically binds the protein or another biomolecule that that exhibits abnormal production, aggregation, and/or deposition.

46. The method of claim 44, wherein the therapeutic agent is selected from a nonspecific clearing antibody (e.g., intravenous immunoglobulin aka IVIg), an anti-amyloid-β antibody (e.g., aducanumab, gantenerumab, lecanemab, and donanemab), an anti-tau antibody (e.g., semorinemab, gosuranemab, tilavonemab, and zagotenemab), an anti-TREM2 antibody (e.g., AL002), an anti-alpha-synuclein antibody (e.g., Cinpanemab, Prasinezumab, Lu AF82422, ABBV-0805, and MEDI1341), and or a combination thereof.

47. The method of claim 41, wherein the therapeutic agent is or comprises a small molecule drug.

48. The method of claim 47, wherein the therapeutic agent provides one or more of synaptic plasticity, neuroprotection, reduction of inflammation, neurotransmitter receptor modulation, reduction of oxidative stress.

49. The method of claim 48, wherein the therapeutic agent is selected from donepezil, galantamine, rivastigmine, memantine, suvorexant, carbidopa-levodopa, selegiline, rasagiline, safinamide, entacapone, benztropine, tolcapone, opicapone, nuplazid, istradefylline and amantadine, and a combination thereof.

50. The method of claim 37, wherein the therapeutic agent is formulated in a liposome.

51. The method of claim 37, wherein the therapeutic agent is delivered via a viral vector.

52. The method of claim 37, further comprising introducing microbubbles into at least one of the target region or its surrounding regions.

53. A method of treating a central nervous system infection in a subject in need thereof, the method comprising:
(a) causing an ultrasound transducer to transmit acoustic energy to a target region at a transmission frequency, wherein the target region encompasses the locus and adjacent blood-brain barrier (BBB);
(b) acquiring a cumulative harmonic response from at least the target region;
(c) operating the transducer based at least in part on the acquired cumulative harmonic response to achieve a target level of BBB permeability; and
(d) administering a therapeutic agent to the target region at least when the target permeability level has been reached.

54. The method of claim 53, wherein the therapeutic agent comprises at least one of an antibiotic, an anti-viral, an anti-retroviral, or an anti-fungal.

55. The method of claim 53, further comprising introducing microbubbles into at least one of the target region or its surrounding regions.

56. A method of treating a congenital enzyme defect in a subject in need thereof, the method comprising:
(a) causing an ultrasound transducer to transmit acoustic energy to a target region at a transmission frequency, wherein the target region encompasses the locus and adjacent blood-brain barrier (BBB);
(b) acquiring a cumulative harmonic response from at least the target region;
(c) operating the transducer based at least in part on the acquired cumulative harmonic response to achieve a target level of BBB permeability; and
(d) administering a therapeutic agent to the target region at least when the target permeability level has been reached.

57. The method of claim 56, wherein the therapeutic agent comprises an enzyme replacement therapy.

58. The method of claim 56, further comprising introducing microbubbles into at least one of the target region or its surrounding regions.
